SEQUENCE LISTING (14 sequences)

SEQ ID NO: 1 - RNA, 9 nt, Escherichia coli
uaaggaggu

SEQ ID NO: 2 - DNA, 324 bp, Artificial sequence
  synthetic MIA gene sequence
atgggcccgatgccgaaactggcggatcgtaaactgtgcgcggatcaggaatgcagccatccgattagcatggcggtggcgctgcaagattacatggcgccggattgccgttttctgaccattcatcgtggccaggtggtgtatgtgtttagcaaactgaaaggccgtggccgtctgttttggggcggcagcgtgcagggcgattactatggcgatctggcggcacgtctgggctatttcccgagcagcattgtgcgtgaagatcagaccctgaaaccgggcaaagtggatgtgaaaaccgacaaatgggatttctattgccag

SEQ ID NO: 3 - DNA, 33 bp, Artificial Sequence
  Synthetic primer used in conjunction with SEQ ID NO 4 to amplify the To terminator by PCR
aaaaagcttgactcctgttgatagatccagtaa

SEQ ID NO: 4 - DNA, 31 bp, Artificial sequence
  Synthetic primer used in conjunction with SEQ ID NO 3 to amplify the To terminator by PCR
aaaacatgtattctcaccaataaaaaacgcc

SEQ ID NO: 5 - DNA, 25 bp, Artificial sequence
  Synthetic primer used in conjunction with SEQ ID NO 6 to amplify the Km cassette (990 bp) from pACYC177 (NEB) by means of PCR
aagggcccgccacgttgtgtgtctc

SEQ ID NO: 6 - DNA, 31 bp, Artificial sequence
  Synthetic primer used in conjunction with SEQ ID NO 5 to amplify the Km cassette (990 bp) from pACYC177 (NEB) by means of PCR
aaagctagcgatatcgccgtcccgtcaagtc

SEQ ID NO: 7 - DNA, 28 bp, Artificial sequence
  Synthetic primer used in conjunction with SEQ ID NO 8 to amplify vector pSCIL001 (1462 bp) without Amp cassette (i.e. the primers used flanked the Amp cassette present in pUC19) by means of PCR
aaagctagcgggaataagggcgacacgg

SEQ ID NO: 8 - DNA, 30 bp, Artificial sequence
  Synthetic primer used in conjunction with SEQ ID NO 7 to amplify vector pSCIL001 (1462 bp) without Amp cassette (i.e. the primers used flanked the Amp cassette present in pUC19) by means of PCR
aaagggcccacgtgagttttcgttccactg

SEQ ID NO: 9 - DNA, 29 bp, Artificial sequence
  Synthetic primer used in conjunction with SEQ ID NO 10 to amplify the proBA operon (2520 bp) by means of PCR
aaagggcccgcaaccgacgacagtcctgc

SEQ ID NO: 10 - DNA, 29 bp, Artificial sequence
  Synthetic primer used in conjunction with SEQ ID NO 9 to amplify the proBA operon (2520 bp) by means of PCR
aaagggccccggtggacaaaggttaaaac

SEQ ID NO: 11 - DNA, 27 bp, Artificial sequence
  Synthetic primer used in conjunction with SEQ ID NO 12 to amplify the lacI gene including the native promoter (1160 bp) by PCR
aaagctagcgacaccatcgaatggcgc

SEQ ID NO: 12 - DNA, 25 bp, Artificial sequence
  Synthetic primer used in conjunction with SEQ ID NO 11 to amplify the lacI gene including the native promoter (1160 bp) by PCR
aaagctagctcactgcccgctttcc

SEQ ID NO: 13 - DNA, 32 bp, Artificial sequence
  Synthetic primer used in conjunction with SEQ ID NO 14 to amplify the tac promoter (67 bp) by means of PCR
aaacaattgtgttgacaattaatcatcggctc

SEQ ID NO: 14 - DNA, 35 bp, Artificial sequence
  Synthetic primer used in conjunction with SEQ ID NO 13 to amplify the tac promoter (67 bp) by means of PCR
aaagaattctctccttgtgaaattgttatccgctc

(19) United States
(12) Patent Application Publication
(10) Pub. No.: US 2007/0015248 A1
(43) Pub. Date: Jan. 18, 2007

(54) Expression vector and use thereof

(76) Inventors: Andreas Anton, Halle (DE); Markus Fiedler, Halle (DE); Andreas Frings, Halle (DE)

Correspondence Address: JENKINS, WILSON, TAYLOR & HUNT, P. A., 3100 TOWER BLVD, SUITE 1200, DURHAM, NC, 27707, US

(21) Appl. No.: 11/471,799

(22) Filed: Jun. 21, 2006

Related U.S. Application Data
(63) Continuation of application No. PCT/EP04/14635, filed on Dec. 22, 2004.

(30) Foreign Application Priority Data
Dec. 22, 2003 (DE) 10360483.9

(51) Int. Cl. C12P 21/06; C12N 1/21; C12N 15/74; C07K 14/52; C07K 14/54; C07K 14/56; C07K 14/51; C07K 14/705; C07K 14/745

(52) U.S. Cl. 435/69.1; 435/471; 435/252.3; 435/472; 530/350; 530/351; 530/303; 530/383; 530/388.1

(57) ABSTRACT

The present invention relates to an expression vector for the use in an auxotrophic, prokaryotic host cell and relates to an expression system containing an expression vector and an auxotrophic, prokaryotic host cell. The invention furthermore relates to an antibiotic-free fermentation medium containing an expression vector as mentioned above as well as to a method for the antibiotic-free expression of peptides/proteins.

### RELATED APPLICATIONS

This application is a continuation of PCT International Patent Application No. PCT/EP2004/014635, filed Dec. 22, 2004, which claims priority to German Patent Application No. 10360483.9, filed Dec. 22, 2003, the disclosures of each of which are incorporated herein by reference in their entitety.

### TECHINICAL FIELD

The present invention relates to an expression vector for the use in an auxotrophic, prokaryotic host cell and relates to an expression system containing an expression vector and an auxotrophic, prokaryotic host cell. The invention furthermore relates to an antibiotic-free fermentation medium containing an expression vector as mentioned above as well as to a method for antibiotic-free expression of peptides/proteins.

### BACKGROUND

The use of expression vectors, for example plasmids, for the expression of for example therapeutic peptides/proteins has been known for a long time. Thus, by large scale production of recombinant proteins for example in E. coli a wide variety of proteins could be made available for biochemical research, biotechnology and even for medical therapy. An example of successful application of this methodology is the bacterial production of an antigen-binding immunoglobulin F_{ab} fragment in medium and high cell densities (Carter et al., 1992; Schiweck and Skerra, 1995). In general, heterologous biosynthesis with respect to the genetechnological production of therapeutic proteins has gained increasing interest in the last decade.

A problem that often encountered in the production on an industrial scale is a reduction in the yield of recombinant protein per cell. One reason for this phenomenon is the loss of functional plasmid from cultures with high cell density due to a segregating or structural instability of genetically safe expression vectors (Corchero and Villaverde, 1998). In contrast to natural vectors bearing ColEI such plasmids lack mobility and distribution functions usually ensuring inheritance to the progeny. Under laboratory conditions, reduced genetic stability is compensated for by antibiotic selection of the bacteria using a resistance marker. It is difficult, however, to maintain this selective pressure under fermentation conditions, and a loss of functional plasmid thus can occur (Zabriskie and Arcuri, 1986; Broesamle et al., 2000).

Another important parameter for the successful production of a recombinant protein in high yield is the selection of an adequate bacterial host strain able to significantly influence the concentration of the gene product synthesized (Sambrook et al., 1989). One of these is for example the E. coli K12 strain JM83 which has been successfully used since many years on a laboratory scale for the expression of antibody fragments via periplasmatic secretion (Skerra et al., 1991; Fiedler and Skerra, 1999). The E. coli K12 strain JM83 is an example of an auxotropic strain, i.e., the strain is unable to produce an essential amino acid by itself. In the case of E. coli K12 strain JM83 the amino acid is proline. This host strain shows a superior functional expression as compared to many other E. coli strains if the protein is secreted into the bacterial periplasm (Skerra 1994 a, b). This proline-auxotropic strain, however, could not be used for fermentation experiments due to its inability to grow on minimal medium. The use of synthetic media for fermentation is generally preferred for an industrial production scale since the growth properties can be controlled in a simple manner by using a defined carbon source, in most of the cases glucose or glycerol (Yee and Blanch, 1992). In the case of JM83 it was found, however, that a supplementation of a glucose/ammonia/mineral salt medium with proline is not feasible since this amino acid is preferably metabolized both as a carbon and as a nitrogen source (Neidhard et al., 1990) and therefore dictates the growth rate.

The proBA locus (Mahan and Csonka, 1983; Deucht et al., 1984) encodes γ-glutamyl kinase (ProB) and glutamate-5-semialdehyde dehydrogenase (ProA) both playing a key role in the proline biosynthetic pathway.

In Gene 274 (2001) 111-118, M. Fiedler and A. Skerra disclose an expression vector, more particularly a plasmid, comprising the following elements operably linked to each other: a repressor for the control of expression (TetR), a promoter for expression (tet), an antibiotic resistance gene for chloramphenicol as well as proBA. The above-mentioned publication, however, does not describe that the plasmid can be used with the amino acid as the only selection means and the only marker. Furthermore, Fiedler et al. do not disclose the use of the above-mentioned plasmid for the production of recombinant proteins in an antibiotic-free fermentation medium.

The use of antibiotics for the selection of plasmids is a significant disadvantage particularly in the preparation of therapeutic proteins. An antibiotic-free process would achieve a higher acceptance by the regulatory authorities (e.g. FDA, EMEA) since the product will be safer for the patient, and because a markedly less cost-intensive process could be devised due to reduced final product analytics (depletion of the antibiotic in the product).

### SUMMARY

Therefore, it is the object of the present invention to provide a method for antibiotic-free expression of peptides/proteins. It is another object of the invention to provide a fermentation medium and an expression system which are suitable for the use in this method.

These objects are achieved by the subject matter of the independent claims. Preferred embodiments are mentioned in the dependent claims.

In the following, the present invention will be explained in more detail with respect to drawings and the accompanying Examples. It should be understood that the scope of the invention is not restricted to these embodiments which are provided for illustration purposes only.

### BRIEF DESCRIPTION OF THE FIGURES

In the Figures, the steps for the construction of pSCIL043 are shown:

FIG. 1: A plasmid map of the pSCIL043 expression vector.

All relevant gene areas were highlighted by colours. The following regions are important for the function of the vector: lacI (repressor, red); Km (antibiotic resistance, green); proBA (selectable marker, yellow); t₀ (terminator, dark green); tac (promoter, blue).

FIG. 2: Restriction of pUC19 with AflIII and HindIII By this restriction non-coding plasmid areas are deleted from pUC19, and two fragments are obtained: a 359 bp fragment irrelevant for function and a 2327 bp fragment representing the remaining vector. The 2327 bp fragment was purified and used in the following, 1: 100 bp marker Invitrogen; 2: AflIII/HindIII restriction of pUC19; 3: pUC19 uncut; 4: 1 Kb marker MBI.

FIG. 3: Amplification of the t₀ terminator from total Lambda DNA

The t₀ terminator (94 bp) was amplified by means of PCR directly from the chromosomal Lambda DNA obtained from MBI-Fermentas.

FIG. 4: Amplification of the Km cassette from pACYC177

pACYC177 was used as a template for the Km cassette. The PCR product was subcloned into pGEM Teasy (Promega) after purification by the Gel Extraction Kit (Qiagen).

FIG. 5: Amplification of pSCIL001 without Amp cassette

To exchange the antibiotics resistances and to introduce two new restriction sites (NheI and ApaI) pSCIL001 was amplified by means of primers flanking the Amp cassette.

FIG. 6: Restriction analysis of pSCIL002

By restriction with ApaI/NheI the Km cassette is again cut out of the vector. By using EcoRI, EcoRV and AflIII for the restrictions the vector is only linearized. 1: 1 kb marker MBI; 2: pSCIL002 ApaI/NheI; 3: pSCIL002 EcoRI; 4: pSCIL002 EcoRV; 5: pSCIL002 AflIII.

FIG. 7: Amplification of the proBA determinant

By means of the above-mentioned primers the proBA determinant could be amplified. The fragment contains the native terminator of the proBA gene.

FIG. 8: Restriction of pSCIL003 with EcoRV

To confirm correct insertion of the proBA determinant in pSCIL002 the resulting plasmid was cut with EcoRV whereby the following fragments should be generated: 2479, 1542 and 999 bp. In case of a negative result for pSCIL002 the vector should only be linearized. 1: 1 kb marker MBI; 2: pSCIL003 EcoRV; 3: pSCIL003 uncut.

FIG. 9: Complementation of the defective proline biosynthetic pathway in JM83 by means of pSCIL003

On complete medium all strains (wild type=XL1Blue) grow equal to the complemented strain (not shown). On mineral salt medium only the strain containing pSCIL003, but not the strain containing pSCIL002 is able to grow.

FIG. 10: Amplification of lacI

By means of the above-mentioned primers the lacI repressor could be amplified directly from K12 chromosomal DNA. The gene was ligated into the subcloning vector pGEM Teasy (Promega) and the integrity of the PCR product was confirmed by restriction analysis and sequencing.

FIG. 11: Cloning of the promoter

By the PCR product one of the two EcoRI restriction sites was destroyed during ligation.

FIG. 12: Cloning of the MIA gene

From the synthesized product the MIA gene was amplified by means of PCR. After subcloning into pGEM Teasy and restriction the fragment was separated by agarose gel electrophoresis and purified. Afterwards the DNA fragment was ligated into pSCIL008^{EcoRI/PstI}.

FIG. 13: Assay for the expression of SP83-043 (JM83 [pSCIL043])

The expression assay for pSCIL043 was performed with JM83 in mineral salt medium. At an OD of 0.8 the induction was performed with 1 mM IPTG. The expression prior to induction as well as during the hours following induction is shown 1: marker 2: SP83-043 (MSM) prior to induction; 3: SP83-043 (MSM) 1 h following induction; 4: SP83-043 (MSM) 2 h following induction; 5. SP83-043 (MSM) 3 h following induction

FIG. 14: Plasmid stability assay for SP83-043

After the expression had been terminated the cells were diluted and plated on solid LB medium. The colonies grown were picked onto LB-Agar without antibiotics and with antibiotics, respectively. All colonies grew on both media resulting in a plasmid stability of 100%. In contrast, a plasmid stability of 12-35% was detected in an expression assay performed in parallel in E. coli BL21.

FIG. 15: Scheme of the construction of pSCIL043

### DETAILED DESCRIPTION

The key feature of the present invention is the generation of an expression system enabling an antibiotic-free fermentation, i.e. protein/peptide expression.

In particular, the present invention relates to the following:

According to a first aspect the invention relates to an expression vector for the use in an auxotrophic, prokaryotic host cell comprising the following components operably linked to each other:
a) a regulatory sequence,
b) a sequence coding for a protein/peptide,
c) a first selectable marker gene, and
d) a second selectable marker gene wherein the marker gene encodes a protein not expressed by the auxotropic host which is necessary for the biosynthesis of an amino acid for which the host cell is auxotrophic,
   wherein a tet promoter is excluded as the regulatory sequence in a).

Preferably, the regulatory sequence is a tac promoter having a ribosomal binding site. Instead of this promoter, however, many other promoters can be used, particularly all promoters on the basis of the lac promoter. Examples for these promoters are the pac, rac, trc, tic promoter. For further information with respect to promoters useful in the context of the present invention see Brosius J. et al. in J Biol Chem. 1985 Mar. 25; 260(6):3539-41, “Spacing of the −10 and −35 regions in the tac promoter. Effect on its in vivo activity.” and Donovan R. S. et al. in J Ind Microbiol. 1996 March; 16(3):145-54, “Review: optimizing inducer and culture conditions for expression of foreign proteins under the control of the lac promoter.”

It is further possible to use the P_{L}, P_{R} promoters from phage Lambda and ara, the arabinose promoter, which are well-known in the field of molecular biology.

According to one embodiment the expression vector according to the invention has a terminator for the termination of transcription for which the use of the t₀ terminator from bacteriophage Lambda is particularly preferred. In principle, any functional terminator can be used in this position. Numerous examples exist therefor since most operons or genes are flanked by a terminator structure to prevent read-through by the polymerase.

The expression vector according to the invention furthermore carries a repressor gene for which the lacI gene is particularly preferred. A repressor gene as used herein comprises any gene encoding a protein which prevents the transcription of genes after binding to the promoter region. An example according to the invention of these is the above-mentioned lac repressor gene (particularly the lac repressor gene from E. coli K12; see examples).

Another well-known repressor is the CI repressor from phage Lambda which, however, does not bind to the lac hybrid promoters but only to the P_{L} and P_{R} promoters. Furthermore, the AraC repressor can be used since it suppresses transcription from the pBAD (ara) promoter. Numerous other examples of repressors which can be employed in the same way are known to those skilled in the art.

Sequence information in this respect can be found for example in the following references
for CI:
Sauer R T, DNA sequence of the bacteriophage gama cI gene. Nature. 1978 Nov. 16; 276(5685):301-2.
Humayun, Z. DNA sequence at the end of the CI gene in bacteriophage lambda, Nucleic Acids Res. 4 (7), 2137-2143 (1977)
Sequence information and additional references with respect to the repressor can be found at:
http://www.ncbi.nlm.nih.gov/entrez/viewer.fcgi?val=9626243&itemID=48& view=gbwithparts
for P_{L} and P_{R}:
Horn, G. T. and Wells, R. D. The leftward promoter of bacteriophage lambda. Isolation on a small restriction fragment and deletion of adjacent regions, J. Biol. Chem. 256 (4), 1998-2002 (1981)
Remaut, E., Stanssens, P. and Fiers, W. Plasmid vectors for high-efficiency expression controlled by the PL promoter of coliphage lambda, Gene 15 (1), 81-93 (1981)
Petrov, N. A., Karginov, V. A., Mikriukov, N. N., Serpinski, O. I. and Kravchenko, V. V. Complete nucleotide sequence of the bacteriophage lambda DNA region containing gene Q and promoter pR′, FEBS Lett. 133 (2), 316-320 (1981)
Walz, A. and Pirrotta, V. Sequence of the PR promoter of phage lambda; Nature 254 (5496), 118-121 (1975)
Sequence information with respect to the promoters can be found at:
http://www.ncbi.nlm.nih.gov/entrez/query.fcgi?cmd=Retrieve&db=nucleotid e&list_uids=14819
http://www.ncbi.nlm.nih.gov/entrez/query.fcgi?cmd=Retrieve&db=nucleotid e&list_uids=341294
http://www.ncbi.nlm.nih.gov/entrez/query.fcgi?cmd=Retrieve&db=nucleotid e&list_uids=215104
for AraC:
Miyada, C. G., Horwitz, A. H., Cass, L. G., Timko, J. and Wilcox, G. DNA sequence of the araC regulatory gene from Escherichia coli B/r, Nucleic Acids Res. 8 (22), 5267-5274 (1980)
Wallace, R. G., Lee, N. and Fowler, A. V. The araC gene of Escherichia coli: transcriptional and translational start-points and complete nucleotide sequence, Gene 12 (3-4), 179-190 (1980)
Sequence information regarding AraC can be found at:
http://www.ncbi.nlm.nih.gov/entrez/query.fcgi?cmd=Retrieve&db=nucleotid e&list_uids=40935
   for the pBAD promoter:
Miyada, C. G., Sheppard, D. E. and Wilcox, G. Five mutations in the promoter region of the araBAD operon of Escherichia coli B/r, J. Bacteriol. 156 (2), 765-772 (1983)
Sequence information regarding the araBAD promoter can be found at:
http://www.ncbi.nlm.nih.gov/entrez/query.fcgi?cmd=Retrieve&db=nucleotid e&list_uids=145308

According to a preferred embodiment the expression vector according to the invention contains a ribosomal binding site having the sequence AGGAGA.

A ribosomal binding site is a binding site for the small subunit of the ribosome on the mRNA upstream of the start codon. In prokaryotes this generally corresponds to the Shine-Dalgarno (SD) sequence which is often localized three to eleven nucleotides upstream of the start codon and shows complementarity to a region at the 3′ end of the 16sRNA. The Escherichia coli SD consensus sequence is UAAGGAGGU.

According to another embodiment of the expression vector according to the invention the first selectable marker gene is an antibiotic resistance gene, preferably a kanamycin resistance gene. Antibiotic resistance genes which can be used in the present context are for example: ampicillin resistance (amp); tetracycline resistance (tet); chloramphenicol resistance (cat); neomycin resistance (corresponding to the kanamycin resistance gene; e.g. the Km resistance gene from vector pACYC177; see examples).

Information with respect to pACYC177 can be found in: Rose, R. E. The nucleotide sequence of pACYC177, Nucleic Acids Res. 16 (1), 356 (1988), as well as at the link http://www.fermentas.com/techinfo/nucleicacids/sequences/pacyc177.txt.

As described above, the expression vector according to the invention contains a second selectable marker gene wherein the marker gene encodes an amino acid not expressed by the auxotropic host. This second selectable marker gene is also referred to a auxotrophy gene herein. The second selectable marker preferably is proBA, the methionine auxotrophy gene metB and/or the leucine auxotrophy gene leuB.

The following data from the literature with respect to proBA describe the genes and their activity: Baich, A., (1969) Proline synthesis in Escherichia coli. A proline inhibitable-glutamic acid kinase. Biochim Biophys Acta 192, 462-467. Baich, A., (1971) The biosynthesis of proline in Escherichia coli, phosphate-dependent glutamate γ-semialdehyde dehydrogenase (NADP), the second enzyme in the pathway. Biochim Biophys Acta 244, 129-134.

In the following references with respect to metB the gene and its activity have been described: Duchange, N., Zakin, M. M., Ferrara, P., Saint-Girons, I., Park, I., Tran, S. V., Py, M. C. and Cohen, G. N. Structure of the metJBLF cluster in Escherichia coli K12. Sequence of the metB structural gene and of the 5′- and 3′-flanking regions of the metBL operon, J. Biol. Chem. 258 (24), 14868-14871 (1983).

Sequence data can be found at:

http://www.ncbi.nlm.nih.gov/entrez/viewer.fcgi?val=146844&itemID=5&view=gb withparts

Data from the literature concerning leuB in which the gene has been described can be found in: Blattner, F. R., Plunkett, G. III, Bloch, C. A., Perna, N. T., Burland, V., Riley, M., Collado-Vides, J., Glasner, J. D., Rode, C. K., Mayhew, G. F., Gregor, J., Davis, N. W., Kirkpatrick, H. A., Goeden, M. A., Rose, D. J., Mau, B. and Shao, Y. The complete genome sequence of Escherichia coli K-12, Science 277 (5331), 1453-1474 (1997).

Sequence data can be found at:

http://www.ncbi.nlm.nih.gov/entrez/viewer.fcgi?val=1786250&itemID=37&view=g bwithparts

By means of the expression vector according to the invention numerous coding sequences can be expressed which can be chosen without any limitation. The expression vector according to the invention has been found particularly advantageous for the expression of the G-CSF, MIA and/or BMP coding sequences. Furthermore, all therapeutically relevant proteins can be expressed (e.g. tPA, TNF, HGF, NGF, proteases such as trypsin, thrombin, enterokinase, β-TGF, interferons, erythropoietin, insulin, Factor VII, Factor VIII, single chain antibodies, Affilin™ as well as fusions of these proteins, G protein coupled receptors as well as the domains thereof, and the pro-forms of these proteins) to be produced as inclusion bodies or soluble variations thereof. Finally, also the expression of GM-CSF, M-CSF, interleukins, interferons, calcitonin, caspases, VEGF, Factor III, Factor X, Factor Xa, Factor XII, Factor XIIa, GDF, IGF, metalloproteases, antibodies, antibody fragments or immunotoxins can be considered.

According to another embodiment the vector according to the invention is a high copy plasmid. A high copy plasmid or high copy number plasmid (also called multi copy plasmid). respectively, refers to small plasmids (usually <15 k b) present in a high copy number (>20 plasmids/chromosome). Such plasmids such as for example pUC plasmids derived from pBR322 are often employed as cloning and expression vectors.

In a specific embodiment the expression vector according to the invention contains the following components operably linked to each other:
a) a tac promoter having a ribosomal binding site,
b) a coding sequence for e.g. MIA,
c) a kanamycin resistance gene,
d) a proBA sequence,
e) optionally the lacI repressor gene, and
f) optionally the tₒ terminator from Lambda for termination of the transcription of a gene.

Furthermore, the invention relates to the expression vector pSCIL008 containing the following components operably linked to each other:
a) a tac promoter having a ribosomal binding site,
b) a coding sequence for e.g. MIA, G-CSF, ProBMP, BMP, tPA, TNF, HGF, NGF, proteases such as trypsin, thrombin, enterokinase, β-TGF, interferons, erythropoietin, insulin, Factor VII, Factor VIII, single chain antibodies, Affilin™ as well as fusions of these proteins, G protein coupled receptors as well as the domains thereof, and the pro-forms of these proteins, GM-CSF, M-CSF, interleukins, interferons, calcitonin, caspase, VEGF, Factor III, Factor X, Factor Xa, Factor XII, Factor XIIa, GDF, IGF, metalloproteases, antibodies, antibody fragments or immunotoxins,
c) an antibiotic resistance gene, preferably a kanamycin resistance gene,
d) a proBA sequence,
e) optionally a repressor binding to the operator of the promoter, preferably the lacI repressor gene, and
f) optionally the tₒ terminator from Lambda for termination of the transcription of a gene.

According to a second aspect the present invention relates to an expression system comprising the following components:
a) an expression vector as defined herein, and
b) an auxotropic prokaryotic host cell.

According to the invention the host cell preferably is an auxotropic E. coli cell which is auxotrophic for the amino acid proline.

The E. coli cell preferably is chosen among the strains JM106, JM108, JM109, JM83 and TB1 or the derivatives thereof. Derivatives is intended to mean strains which were genetically manipulated but retain the auxotrophies relevant for expression and thus can be transformed by the vectors according to the invention which complement the amino acid auxotrophies. The invention also comprises those strains that were manipulated by methods according to the prior art to acquire auxotrophies which can be used as selectable markers.

The above-mentioned E. coli strains have been deposited under the following DSMZ numbers:

JM83 DSMZ no. 3947

JM108 DSMZ no. 5585

JM109 DSMZ no. 3423

A detailed description of JM106 can be found in Yanisch-Perron et al., 1985. Information for TB-1 is available in Biochemistry 23:3663-3667 1984.

According to a third aspect the invention relates to an antibiotic-free fermentation medium comprising the following components:
a) an expression system as defined above, or
b) an expression system containing
   aa) an expression vector having the following components:
      a regulatory sequence,
      a sequence coding for a protein/peptide,
      a first selectable marker gene, and
      a second selectable marker gene wherein the marker gene encodes an amino acid not expressed by the auxotropic host,
      optionally a terminator for termination of the transcription.
   bb) an auxotropic prokaryotic host cell,
c) a suitable aqueous mineral salt medium, and
d) in the presence of repressor genes optionally an inductor.

According to a forth aspect the present invention relates to a method for antibiotic-free expression of peptides/proteins comprising the die following steps:
a) transforming auxotropic host cells with an expression vector as defined herein,
b) selecting transformed host cells wherein the selection is performed on the basis of the amino acid expressed by the second selectable marker gene;
c) introducing the transformed host cells into a fermentation medium as described above under conditions that fermentation occurs and the protein/peptide is expressed; and
d) isolating and purifying the expressed protein/peptide.

According to one embodiment the selection in step b) is additionally carried out by an antibiotic.

In other words, in the present invention particularly the fermentation process, i.e. the actual process stage for the expression of the peptides/proteins, is performed in a completely antibiotic-free environment. In this manner, the problems mentioned in the beginning which accompany the use of antibiotics for the selection of plasmids such as for example concerns of the regulatory authorities, product safety, final product analytics (depletion of the antibiotic in the product) and the risks and costs associated therewith can be circumvented. By the approach according to the invention the selective pressure during the fermentation process is still maintained, and this without using antibiotics in the fermentation medium.

### EXAMPLES

### Example 1

### Description of Cell Line SP83-043 [JM83(pSCIL043]

1) Origin, Phenotype and Genotype of the Expression Strain
The bacterial host Escherichia coli JM83 used for the expression of MIA was obtained from the Deutsche Sammlung für Mikroorganismen und Zellkulturen GmbH, Braunschweig (DSMZ) and is identified by a certificate.
The E. coli strain JM83 (DSMZ 3947) [F⁻, ara, Δ(lac-proAB), rpsL (Str^{r}), φ80lacZΔM15, thi] used is resistant to streptomycin due to a mutation in the rpsL gene (12 S protein of the 30 S subunit of the bacterial ribosome). Due to a mutation in the proBA operon the strain is unable to synthesize proline. This effect, however, is abolished by using pSCIL043, and this auxotrophy is utilized as selectable marker. Furthermore, the strain is unable to metabolize arabinose and like many other K12 derivatives (e.g. C600; DH5α, JM107) cannot synthesize thiamine (Vieira & Messing, 1982).

2) Expression of MIA
The MIA protein is expressed in the Escherichia coli strain JM83 under the control of the tac promoter localized on pSCIL043. The pSCIL043 vector is a high copy plasmid bearing a kanamycin resistance. The expression is performed in defined mineral salt medium and is induced by addition of IPTG. MIA is synthesized in the form of so-called inclusion bodies.

3) References
Vieira, J. and Messing, J. (1982); Gene 19, p. 259
JM83 (DSMZ no. 3947)

MIA gene sequence (synthetic gene):

ATGGGCCCGATGCCGAAACTGGCGGATCGTAAACTGTGCGCGGATCAGGA
ATGCAGCCATCCGATTAGCATGGCGGTGGCGCTGCAAGATTACATGGCGC
CGGATTGCCGTTTTCTGACCATTCATCGTGGCCAGGTGGTGTATGTGTTT
AGCAAACTGAAAGGCCGTGGCCGTCTGTTTTGGGGCGGCAGCGTGCAGGG
CGATTACTATGGCGATCTGGCGGCACGTCTGGGCTATTTCCCGAGCAGCA
TTGTGCGTGAAGATCAGACCCTGAAACCGGGCAAAGTGGATGTGAAAACC
GACAAATGGGATTTCTATTGCCAG

Detailed Description of the pSCIL043 Expression Vector

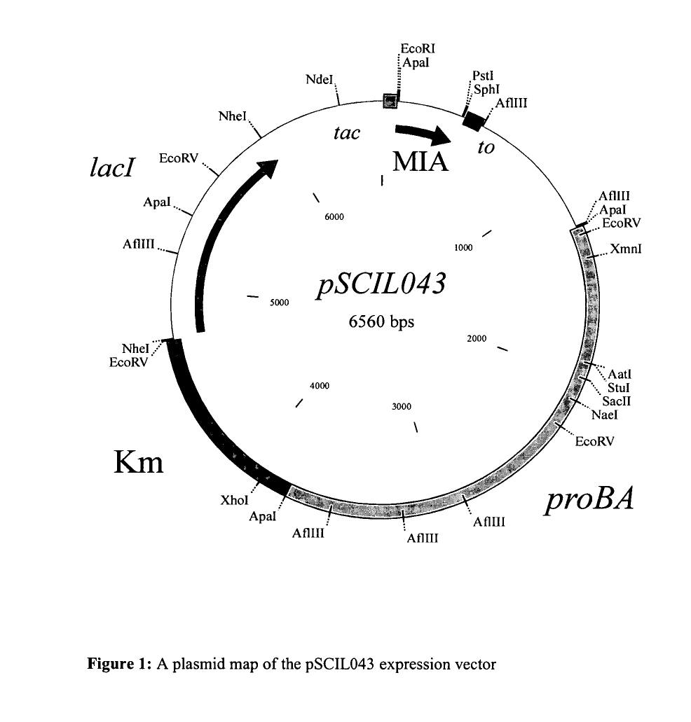

In the following, a detailed description of plasmid pSCIL043 is given. The MunI/EcoRI site (G¹AATTG) which is no longer intact due to the cloning strategy is counted as the first base. A plasmid map is shown in FIG. 1:

| position (bp) | function/description |
|---|---|
| 1-730 | region relevant for expression |
| 7-74 | tac promoter including RBS (AGGAGA) |
| 75-80 | EcoRI restriction site (used for cloning) |
| 83-406 | hMIA gene |
| 407-412 | TAATGA stop codons |
| 413-418 | PstI restriction site (used for cloning) |
| 425-430 | HindIII restriction site (used for cloning) |
| 431-525 | t₀ terminator of bacteriophage Lambda |
| 526-531 | AflIII restriction site (used for cloning) |
| 532-1232 | origin of replication of pUC19 |
| 1233-5929 | selectable marker |
| 1233-1238 | ApaI restriction site (used for cloning) |
| 1239-3759 | proBA determinant as an ApaI/ApaI fragment |
| | (selectable marker) from E coli K12 |
| 3760-3765 | ApaI restriction site (used for cloning) |
| 3766-4756 | Km resistance gene from vector pACYC177 |
| 4757-4762 | NheI restriction site (used for cloning) |
| 4763-5923 | lacI repressor gene as an NheI fragment from E coli |
| | K12 |
| 5924-5929 | NheI restriction site (used for cloning) |
| 5930-6560 | pUC19 backbone |

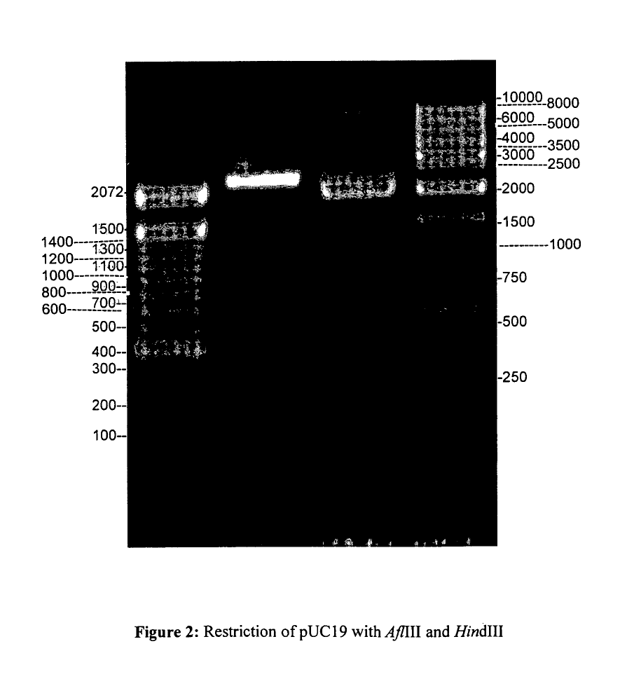

Preparation of the Production Plasmid pSCIL043
1. Insertion of a Transcription Terminators into the Starting Vector=pSCIL001
1.1. Restriction of the Starting Vector
Starting plasmid=pUC19 plasmid obtained from MBI-Fermentas
restriction of the pUC19 plasmid with HindIII and AflIII (FIG. 2) whereby 359 bp are deleted from the vector.
   1.2. Amplification of the t₀ Terminator

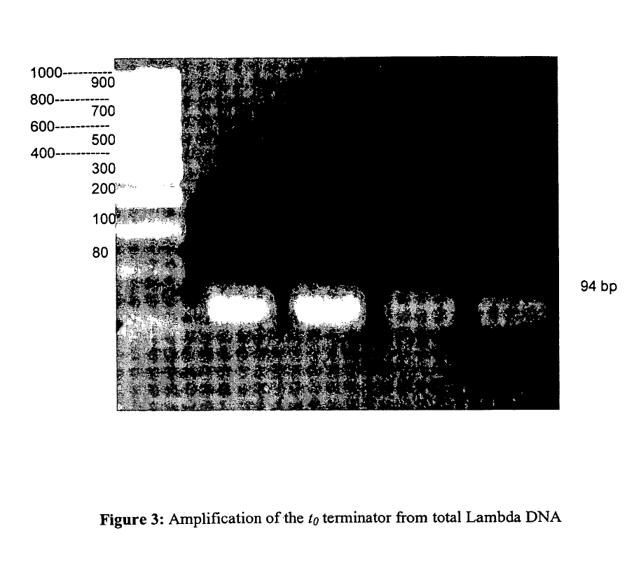

Amplification of the t₀ terminator by means of PCR using the following primers (FIG. 3):

| | |
|---|---|
| 1) | t₀-OD-MCS-HindIII |
| | 5′-AAAAAGCTT^{HindIII}GACTCCTGTTGATAGATCCAGTAA-3′ |
| 2) | t₀-UU-MCS-AflIII |
| | 5′-AAAACATGT^{AflIII}ATTCTCACCAATAAAAAACGCC-3′ |

Restriction of the terminator fragment with restriction HindIII (MBI) and AflIII (NEB) endonucleases and subsequent purification of the fragment using the Minelute Kit (Qiagen)
Ligation of the t₀ terminator into pUC19^{HindIII/AflIII}. Verification of insertion by restriction analysis and sequencing=pSCIL001.

Exchange of the Antibiotic Resistance in pSCIL001=pSCIL002

1.2. Amplification of the Kanamycin Cassette

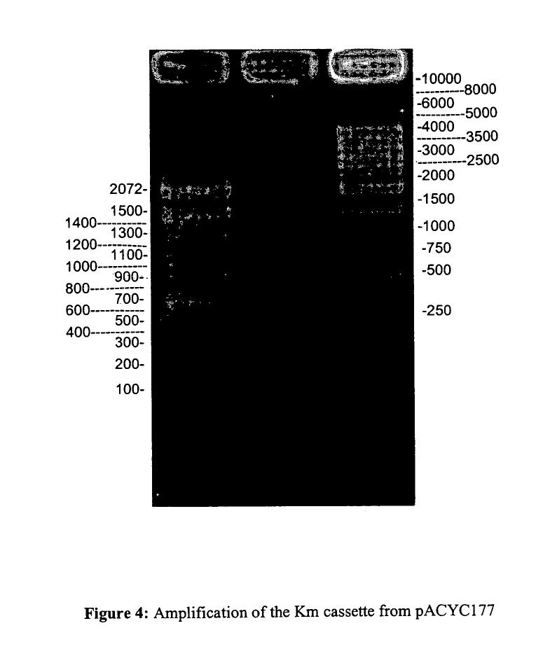

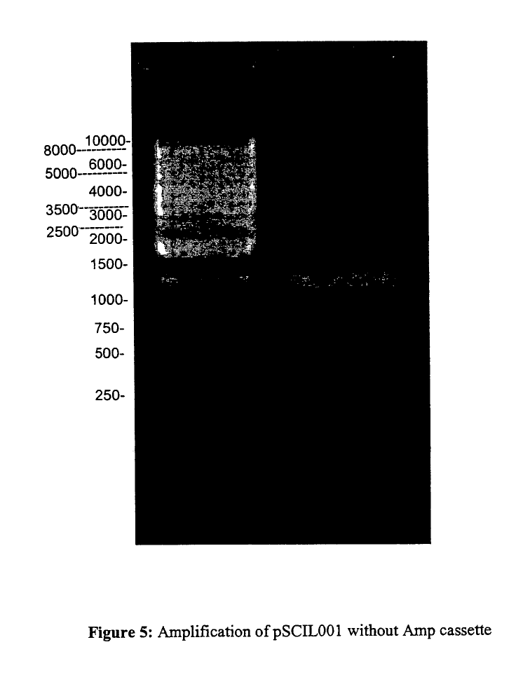

Amplification of the Km cassette (990 bp) from pACYC177 (NEB) by means of PCR using the following primers (FIG. 4):

| | |
|---|---|
| 1) | Km-OD-ApaI |
| | 5′-AAGGGCCC^{ApaI}GCCACGTTGTGTGTCTC-3′ |
| 2) | Km-UU-NheI |
| | 5′-AAAGCTAGC^{NheI}GATATCGCCGTCCCGTCAAGTC-3′ |

Subcloning of the PCR product into pGEM Teasy (Promega). The integrity of the fragment was examined by restriction analysis and sequencing of the DNA.
   2.2. Amplification of pSCIL001 Without Ampicillin Resistance Cassette

Amplification of vector pSCIL001 (1462 bp) without Amp cassette (i.e. the primers used flanked the Amp cassette present in pUC19) by means of PCR using the following primers (FIG. 5):

| | |
|---|---|
| 1) | pUC2451-OD-NheI |
| | 5′-AAAGCTAGC^{NheI}GGGAATAAGGGCGACACGG-3′ |
| 2) | pUC1496-UU-ApaI |
| | 5′-AAAGGGCCC^{APaI}ACGTGAGTTTTCGTTCCACTG-3′ |

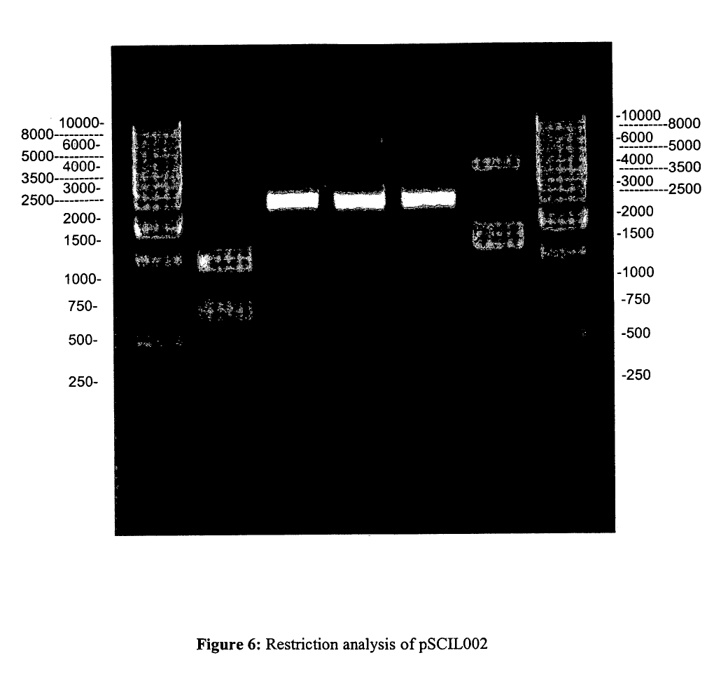

The Km cassette present in the subcloning vector pGEM Teasy was cut out from the vector by restriction with ApaI/NheI and was ligated to the already cut pSCIL001(ΔAmp)^{ApaI/NheI} fragment (FIG. 5.). This resulted in pSCIL002.
pSCIL002 was examined by means of restriction analysis. Four different restrictions (ApaI/NheI, EcoRI, EcoRV and AflIII) were performed on an analytic scale. In all restrictions the vector should be linearized, only the treatment with ApaI and NheI should result in release of the inserted Km cassette (FIG. 6.).
   3. Insertion of the Secondary Selectable Marker in pSCIL002=pSCIL003
   3.1. Amplification of the proBA Determinant from K12 Chromosomal DNA

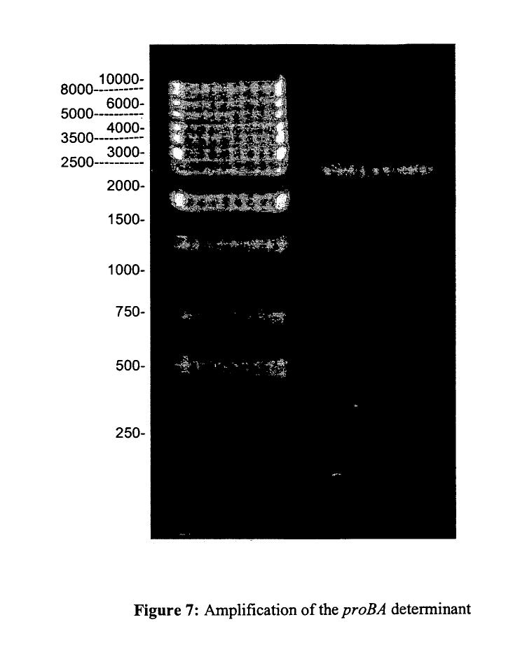

Amplification of the proBA operon (2520 bp) by means of PCR was done using the following primers (FIG. 7). K12 chromosomal DNA was used as a template for the PCR. This DNA was isolated by means of the DNeasy Tissue Kit (Qiagen). The E. coli K12 strain (DSMZ 9037) was obtained from DSMZ, Braunschweig:

| | |
|---|---|
| 1) | proAB-OD-ApaI |
| | 5′-AAAGGGCCC^{APaI}GCAACCGACGACAGTCCTGC-3′ |
| 2) | proAB-UU-ApaI |
| | 5′-AAAGGGCCC^{APaI}CGGTGGACAAAGGTTAAAAC-3′ |

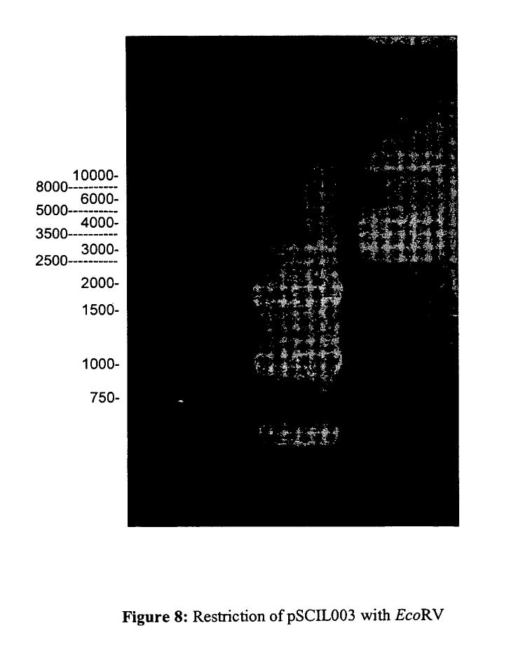

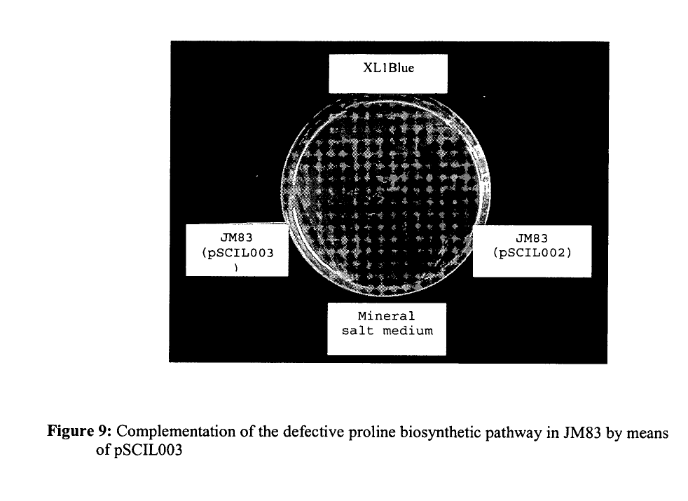

3.2. Cloning of the proBA Determinant into pSCIL002^{ApaI} and Functional Assay
The subcloned proBA fragment was cut out of the pGEM Teasy vector (Promega) by restriction with ApaI and was ligated into vector pSCIL002^{ApaI}. To confirm correct insertion of the secondary selectable marker the pSCIL003 vector was cut with EcoRV (NEB) (FIG. 8).
To test the functionality of the pSCIL003 vector, E. coli strain JM83 was transformed with the vectors pSCIL002 (without proBA) and pSCIL003 (with proBA), respectively. The resulting transformants were plated on mineral salt medium and tested for their ability to grow on this medium (FIG. 9.). It is clear that due to the transformation with pSCIL003 JM83 is able to grow on mineral salt medium. Without the proBA determinant provided on a plasmid the strain is unable to grow on that medium. Therefore, proBA can be employed as a selectable marker.
   4. Insertion of the lacI Repressor=pSCIL004a
   4.1. Amplification of the lacI Repressor from K12 Chromosomal DNA

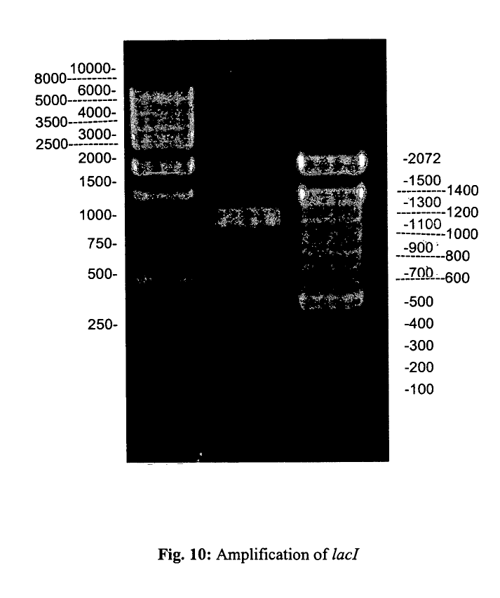

Amplification of the lacI gene including the native promoter (1160 bp) by PCR was performed using the following primers (FIG. 10). K12 chromosomal DNA was used as a template for the PCR. This DNA was isolated by means of the DNeasy Tissue Kit (Qiagen). The E. coli K12 strain (DSMZ 9037) was obtained from DSMZ Braunschweig:

| | |
|---|---|
| 1) | lacI OD NheI |
| | 5′-AAAGCTAGC^{NheI}GACACCATCGAATGGCGC-3′ |
| 2) | lacI UU NheI |
| | 5′-AAAGCTAGC^{NheI}TCACTGCCCGCTTTCC-3′ |

4.2. Introduction of the Repressor Gene into pSCIL003=pSCIL004a
The repressor gene was introduced into pSCIL003 via the NheI restriction site. Since there were two possibilities for the introduction of the repressor gene the exact orientation of the fragment was examined by means of restriction analysis. By means of EcoRV digestion the orientation of the gene in the direction of expression of the later target gene within the MCS could be confirmed (results not shown).
   5. Insertion of the tac Promoter into pSCIL004a=pSCIL008
   5.1. Amplification of the tac Promoter and Cloning Strategy

For the amplification of the tac promoter (67 bp) by means of PCR the following primers were used. Vector pKK233-3 (Amersham, see Annex) was used as a template for the PCR.

| | |
|---|---|
| 1) | Prtac-MunI5′ |
| | 5′-AAACAATTG^{MunI}TGTTGACAATTAATCATCGGCTC-3′ |
| 3) | Prtac-EcoRI3′ |
| | 5′-AAAGAATTC^{EcoRI}TCTCCT^{RBS}TGTGAAATTGTTATCCGCT |
| | C-3′ |

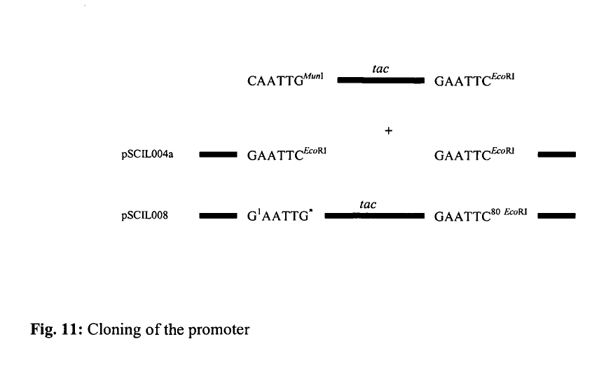

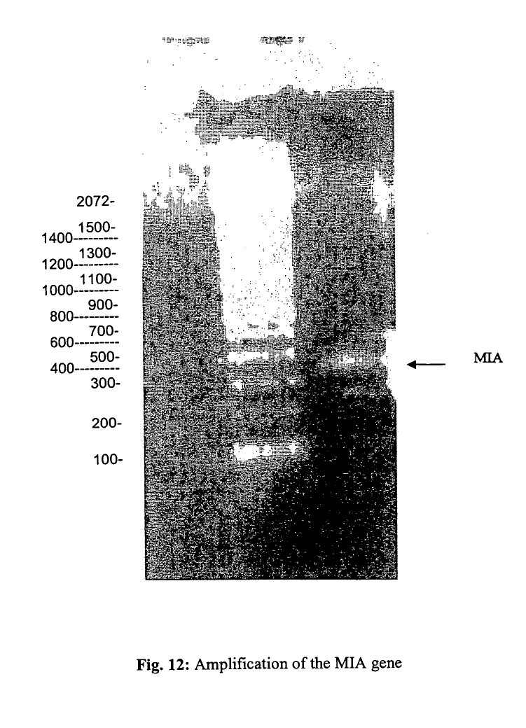

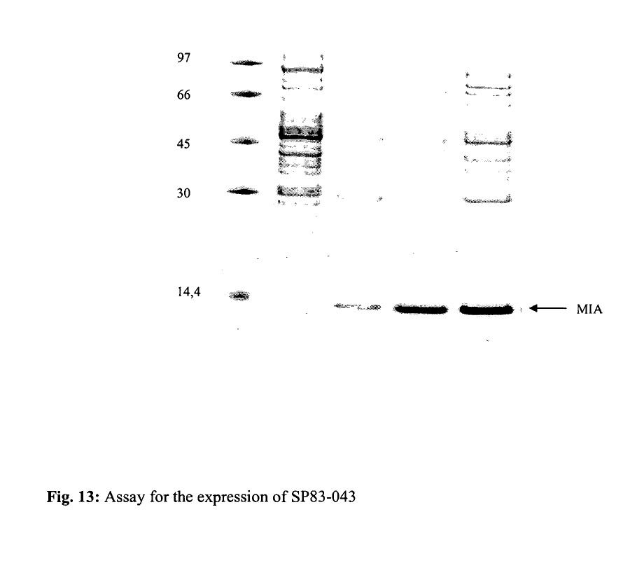

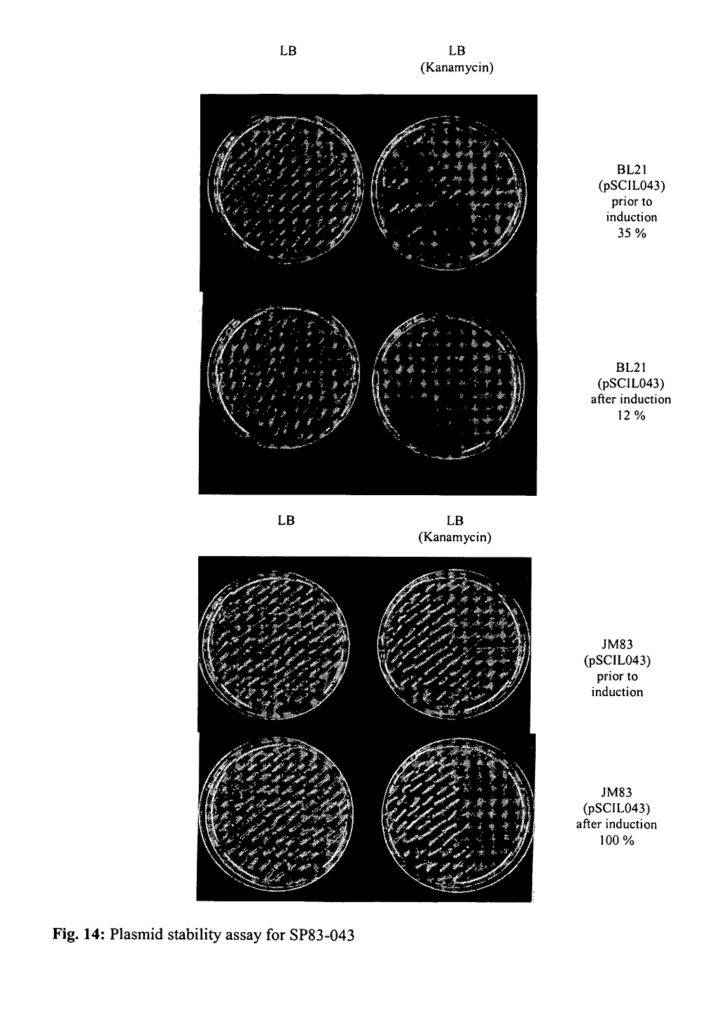

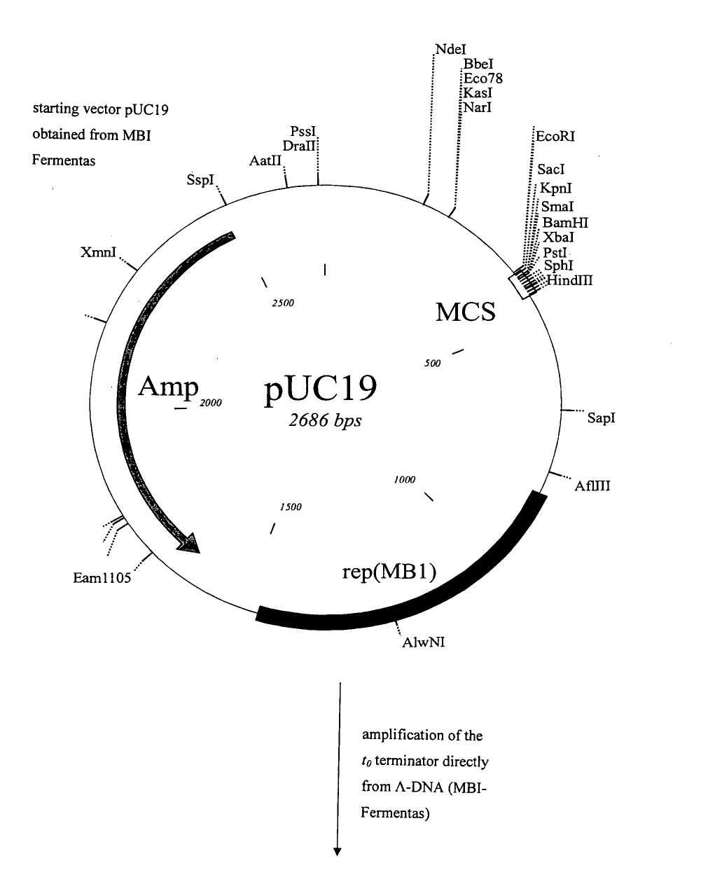

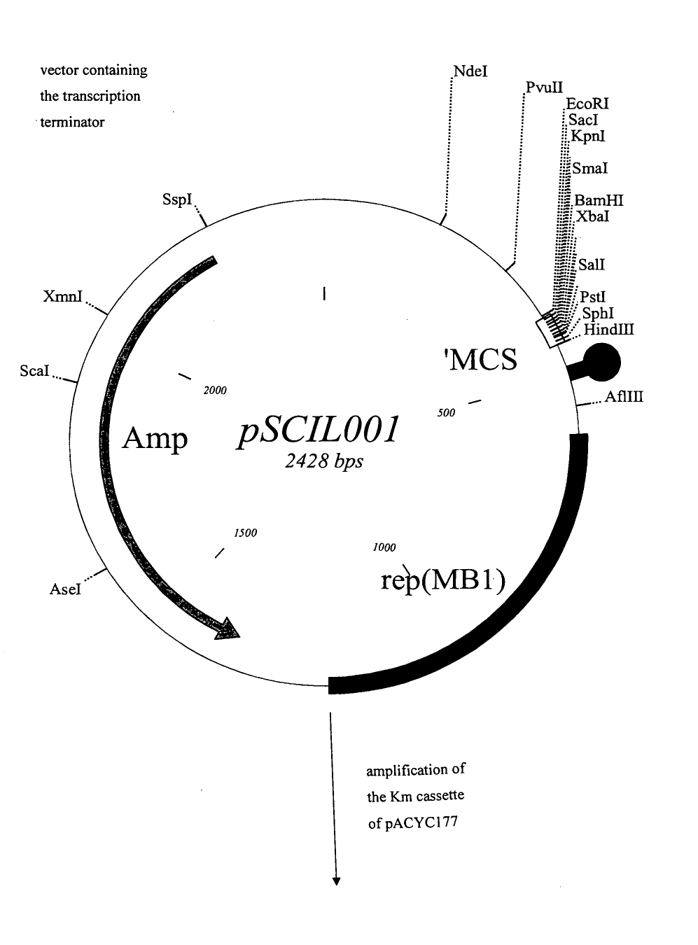

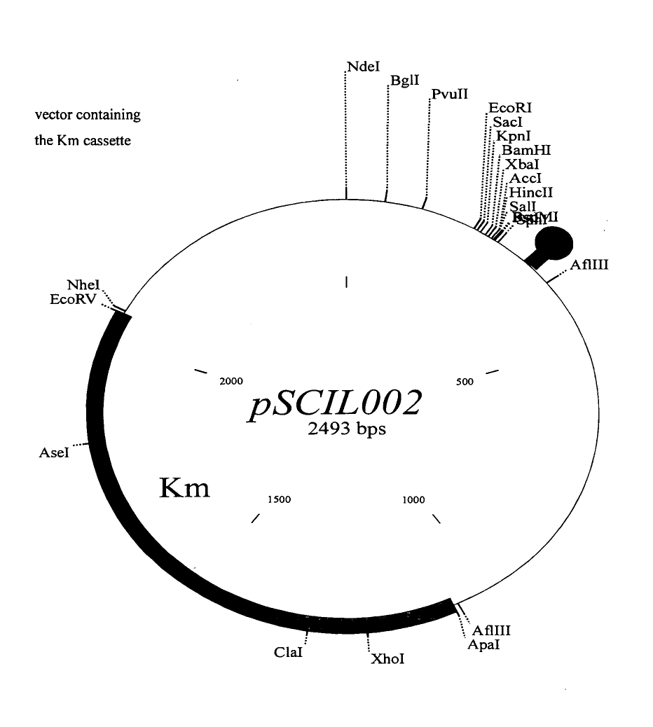

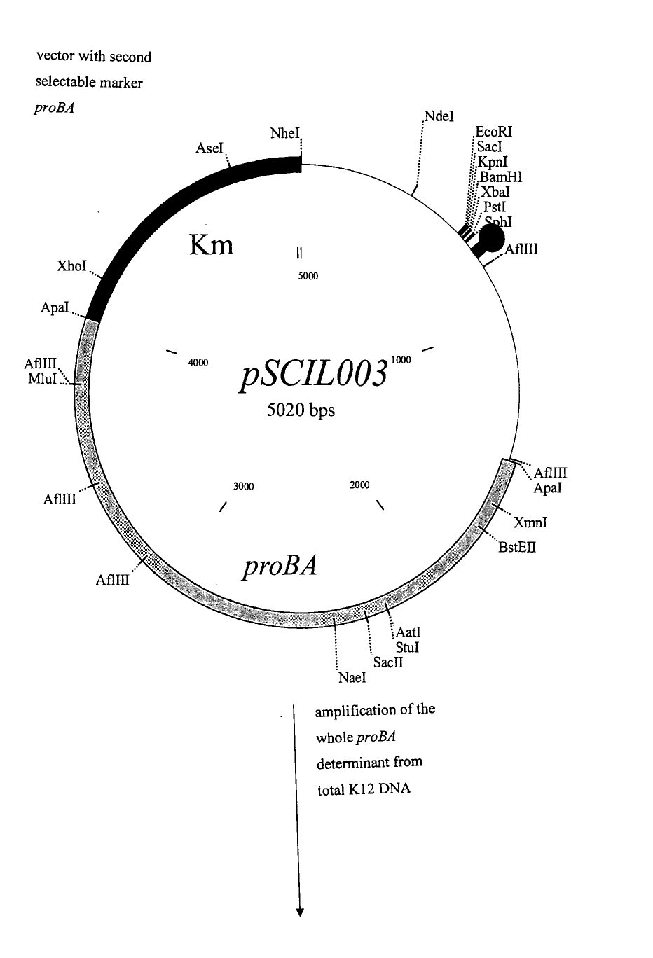

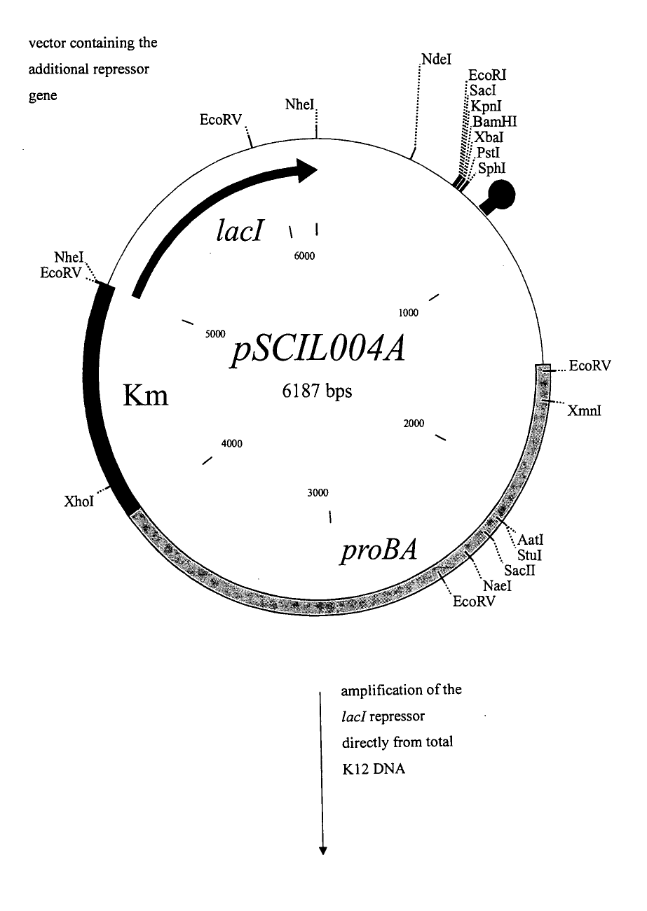

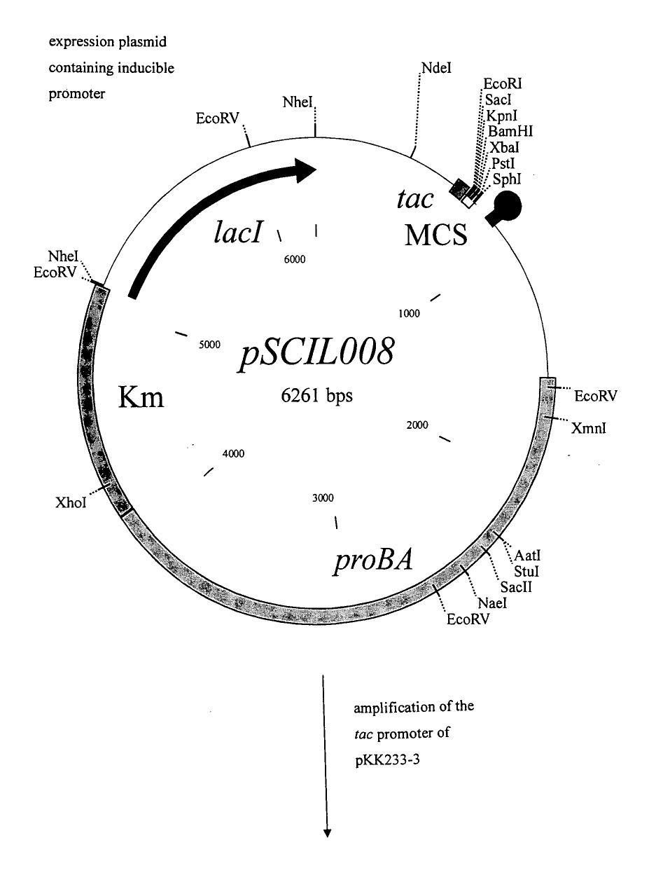

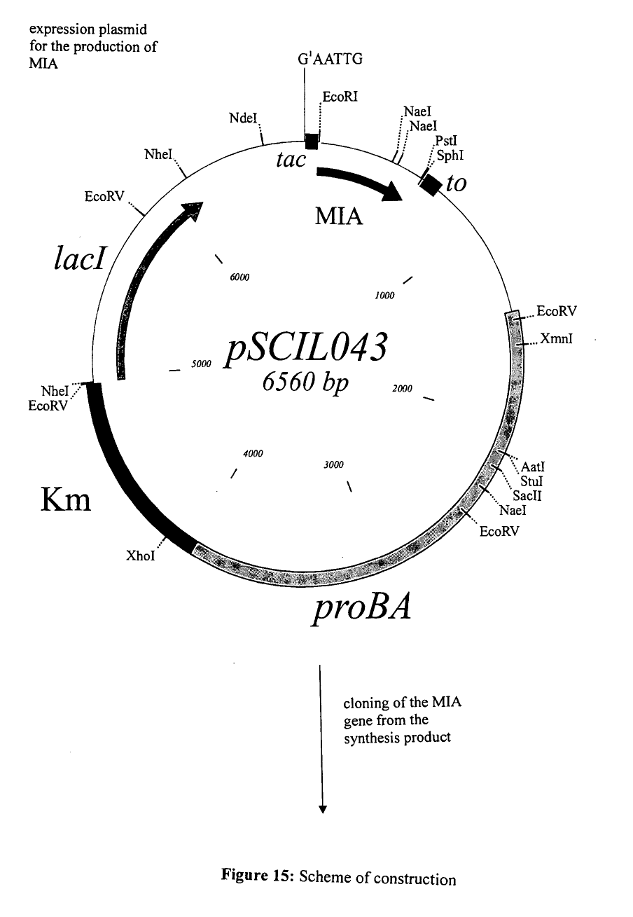

The PCR product was directly treated with the restriction endonucleases EcoRI and MunI. The 3′ primer Prtac EcoRI contains a ribosomal binding site besides the EcoRI site. By ligation into EcoRI cut pSCIL004^{EcoRI} the promoter could be cloned directly upstream of the EcoRI site into the MCS. In this way the second EcoRI site is destroyed by the MunI site at the 5′ end of the promoter. The introduction was confirmed by sequencing (FIG. 11).
   6. Ligation of the MIA Gene
The MIA gene was synthesized by geneART GmbH, Regensburg
The MIA fragment was cut from the synthesis plasmid (FIG. 12) with the restriction endonucleases EcoRI and PstI and was ligated into vector pSCIL008 which was cut by the same enzymes. The introduction was examined by restriction and sequencing. By cloning the MIA gene into the pSCIL008 plasmid the expression vector pSCIL043 was obtained.
E. coli strain JM83 was transformed with the pSCIL043 expression plasmid and adapted to mineral salt medium by repeated plating onto solid media. In an expression experiment (20 ml) the expression performance of the vector was examined (FIG. 13).
Plasmid stability was checked by testing the cells at the end of induction on selective and non-selective (containing kanamycin) LB agar. Thereby, a stability of 100% was obtained (FIG. 14).
   7. Construction Scheme
   (FIG. 15)

### Example 2

### Example of the Fermentation Process

An expression vector according to the invention is transformed into competent E. coli JM83 and E. coli BL21 cells using methods corresponding to the prior art. These cells are first plated on LB agar and incubated at 37° C. Afterwards they must be adapted to mineral salt medium. For this purpose a clone is transferred from the LB agar plate onto a plate containing mineral salt medium agar and is incubated at 37° C. To improve the adaptation to this medium a clone from the agar plate containing mineral salt medium is transferred to another agar plate with mineral salt medium and is incubated at 37° C. 100 ml of mineral salt medium are inoculated with a clone from this plate. The incubation is performed at 180 rpm at 37° C. overnight up to an optical density of OD₆₀₀=3. At this OD glycerol cultures are prepared from the liquid culture which are composed of 800 μl of cell suspension and 200 μl glycerol.

In this Example two fermentations are described in 10 1 laboratory fermenters (B. Braun Biotech). The first is processed with E. coli JM83. The second is performed with E. coli BL21 in which the selectable marker is inactive since BL21 is not auxotrophic. The fermentations are performed as fed batch processes. The batch volume is 6 1. 2 1 of substrate (feed) are dosed to obtain a final volume of 8 1.

As inoculate one glycerol culture in 100 ml of mineral salt medium is incubated overnight at 180 rpm and 37° C. (1^{st} pre-culture). Four flasks with 100 ml of mineral salt medium are inoculated with a partial volume of the first pre-culture and incubated at 180 rpm and 37° C. (2^{nd} pre-culture). At an optical density of e.g. OD₆₀₀=3 the cell suspension is centrifuged and the cell pellet is resuspended in 50 ml of physiological saline.

The batch phase starts with inoculation and ends at a defined optical density, e.g. OD₆₀₀=18, with the start of the fed batch phase, i.e. with the start of substrate dosing. Dosing of the substrate (feed flow) is performed according to an exponential function of the form feed flow=const*exp(μ₁*t) wherein μ is the specific growth rate and wherein μ₁=const<μₘₐₓ. I.e. the amount of substrate at each point of time is always lower than the maximum demand of the cells at this time thereby achieving a submaximal growth rate. For example, μ₁=0.35 h⁻¹ is chosen.

At a defined optical density, e.g. OD₆₀₀=75, the protein expression is induced by addition of IPTG, e.g. 1 mM. At this time another specific growth rate μ₂ is adjusted for the cells, e.g. μ₂=0.1 h⁻¹, by means of substrate dosing (feed flow) The process is terminated after a defined incubation period, e.g. 4 h.

The oxygen demand which continues to increase with increasing cell density is kept constant at e.g. 20% saturation using a cascade control for the oxygen partial pressure pO₂. With increasing demand this results in an increase in agitator rotational speed. If a maximum rotational speed is obtained the gassing rate with air is increased. If the gassing rate reaches its maximum, pure oxygen is dosed with the incoming air.

Nitrogen is supplied via pH regulation wherein ammonia is used as base. Phosphoric acid serves as acid. An anti-foaming agent is automatically dosed in the case of strong foam formation.

Samples for the determination of plasmid stability are retrieved at different time points of fermentation. While a total loss of plasmid and thereby of protein expression is very rapidly encountered in BL21, 100% plasmid stability can be detected in JM83 during the whole fermentation process. In parallel to these samples the plasmids are isolated from several samples (in hourly intervals in the course of the fermentation). The plasmid integrity in JM83 is determined by means of different endonucleases and is found to be unaffected over the whole fermentation process. The restriction patterns showed the same bands as in the starting plasmid.

TABLE 1
Plasmid stabilities

| Time | JM83 (pSCIL043) | BL21 (pSCIL043) |
|---|---|---|
| prior to induction | 100% | 35% |
| following induction | 100% | 12% |

1. An expression vector for the use in an auxotrophic, prokaryotic host cell comprising the following components operably linked to each other:
a) a regulatory sequence,
b) a sequence coding for a protein/peptide,
c) a first selectable marker gene, and
d) a second selectable marker gene wherein the marker gene encodes a protein not expressed by the auxotropic host which is necessary for the biosynthesis of an amino acid for which the host cell is auxotrophic, wherein the regulatory sequence is a tac promoter containing a ribosomal binding site and the second selectable marker is proBA.

2. The expression vector according to claim 1 furthermore containing a terminator for termination of the transcription.

3. The expression vector according to claim 1 further containing a repressor gene.

4. The expression vector according to claim 1 wherein the repressor gene is a lacI gene.

5. The expression vector according to claim 1 wherein die ribosomal binding site has the sequence AGGAGA.

6. The expression vector according to claim 1 wherein the first selectable marker gene is an antibiotic resistance gene, preferably a kanamycin resistance gene.

7. The expression vector according to claim 1 wherein die coding sequence for MIA, G-CSF, ProBMP, BMP, tPA, TNF, HGF, NGF, proteases such as trypsin, thrombin, enterokinase, β-TGF, interferons, erythropoietin, insulin, Factor VII, Factor VIII, single chain antibodies, Affilin™ as well as fusions of these proteins, G protein coupled receptors as well as the domains thereof and pro-forms of these proteins are used.

8. The expression vector according to claim 1 wherein the terminator is tₒ from bacteriophage Lambda.

9. The expression vector according to claim 1 wherein the expression vector is a high copy plasmid.

10. The expression vector pSCIL008 according to claim 1 containing the following components operably linked to each other:
a) a tac promoter having a ribosomal binding site,
b) a coding sequence for e.g. MIA, G-CSF, ProBMP, BMP, tPA, TNF, HGF, NGF, proteases such as trypsin, thrombin, enterokinase, β-TGF, interferons, erythropoietin, insulin, Factor VII, Factor VIII, single chain antibodies, Affilin™ as well as fusions of these proteins, G protein coupled receptors as well as the domains thereof, and the pro-forms of these proteins, GM-CSF, M-CSF, interleukins, interferons, calcitonin, caspase, VEGF, Factor III, Factor X, Factor Xa, Factor XII, Factor XIIa, GDF, IGF, metalloproteases, antibodies, antibody fragments or immunotoxins,
c) an antibiotic resistance gene, preferably a kanamycin resistance gene,
d) a proBA sequence,
e) optionally a repressor binding to the operator of the promoter, preferably the lacI repressor gene, and
f) optionally the tₒ terminator from Lambda for termination of the transcription of a gene.

11. An expression system comprising the following components:
a) an expression vector according to claim 1, and
b) an auxotropic prokaryotic host cell.

12. The expression system according to claim 11 wherein the host cell is an auxotropic E. coli cell which is auxotrophic for the amino acid proline.

13. The expression system according to claim 12 wherein the E. coli cell is selected from the strains JM106, JM108, JM109, JM83 and TB1 or the derivatives thereof.

14. An antibiotic-free fermentation medium comprising an expression system according to claim 11.

15. A fermentation medium according to claim 14 further comprising an inductor in the presence of a repressor gene.

16. A method for antibiotic-free expression of peptides/proteins comprising the following steps:
a) transforming auxotropic host cells with an expression vector according to claim 1,
b) selecting for transformed host cells wherein the selection is performed on the basis of the amino acid expressed by the second selectable marker gene;
c) introducing the transformed host cells into an antibiotic-free fermentation medium according to claim 14 under conditions that fermentation occurs and the protein/peptide is expressed; and
d) isolating and purifying the expressed protein/peptide.

17. The method according to claim 16 wherein the selection in step b) is additionally carried out by an antibiotic.
